(19) United States
(12) Patent Application Publication
(10) Pub. No.: US 2025/0387257 A1
(43) Pub. Date: Dec. 25, 2025

(54) SYSTEM AND SUIT FOR ASSISTING WITH HOMEOSTASIS INTENDED FOR APPLYING AN ALTERNATING COOLING AND HEATING SEQUENCE

(71) Applicant: SPORTOPIA LMB, Longueau (FR)

(72) Inventor: Ludovic BOULAFRAD, Longueau (FR)

(21) Appl. No.: 18/876,994

(22) PCT Filed: Jun. 20, 2023
(86) PCT No.: PCT/EP2023/066569

§ 371 Date: Dec. 19, 2024

(30) Foreign Application Priority Data
Jun. 21, 2022 (FR) FR2206062

(51) Int. Cl. A61F 7/02; A61B 5/397; A61F 7/00; G16H 40/63

(52) U.S. Cl. CPC A61F 7/02; G16H 40/63; A61B 5/397; A61F 2007/0075; A61F 2007/0093; A61F 2007/0096; A61F 2007/0225

(57) ABSTRACT

The present invention relates to a homoeostatic support system having a flexible support, control electronics and a software application. The support includes at least one flexible heating and cooling means integrated inside the support and at least one temperature sensor intended to be associated with at least one zone of a user's body surface. The control electronics communicate with the support and the application communicating with the control electronics so as to receive information measured by temperature sensor via said control electronics. Instructions for applying an alternating cooling and heating sequence are determined and sent to the control electronics.

### TECHNICAL FIELD

The present invention relates to the field of homoeostasis.

The present invention relates more particularly to a system and a suit for supporting a user's homoeostatic phenomena.

For the purposes of the present invention, throughout the description hereinafter, homoeostasis means the phenomena and processes of regulating a user's physical characteristics, in particular blood flow and respiration rate allowing the regulation of dioxygen, ions or nutrients throughout the body, or muscle contraction producing heat. Such a system and such a suit can thus be used to optimise these phenomena and/or to ensure their proper function.

Thus, the present invention will find many advantageous applications in the field of athlete support, in particular in recovery or rehabilitation support. The present invention will also find applications in other broader fields, particularly in the field of health in general, for post-surgery or convalescence recovery support.

### PRIOR ART

The Applicant observes that, in sports, training load programmes have progressed significantly so as to optimise sports performance. However, responsibility for recovery procedures is often left up to the athlete. However, as muscle recovery is a key factor in sports performance, the lack of regular recovery gradually causes the athlete to be overworked or overtrained.

The Applicant thus observes that different solutions have been envisaged to improve the recovery of athletes, so as to allow the athlete to continue training or maintain a stable competitive condition. Such solutions employ hot and/or cold massage techniques, hyperbaric oxygenation, venous return acceleration, or electrostimulation, so as to accelerate the athlete's overall restoration. Other solutions such as local cryotherapy and cold water plunging are also emerging as means of combating muscle inflammation. To date, these solutions remain incomplete, under development, and do not ensure optimal athlete recovery. The implementation of these solutions is also complex and their use is hence limited to specific conditions of use.

The Applicant therefore proposes that there is currently no satisfactory alternative solution for supporting the athlete's muscle recovery and homoeostasis as much as possible, so as to obtain optimal performance and avoid exertion-related injuries.

### SUMMARY OF THE INVENTION

The aim of the present invention is that of improving the current situation described above.

The aim of the present invention is more particularly that of remedying the above limitations by providing an easy-to-use homoeostatic support system, boosting muscle recovery capabilities as much as possible.

To this end, the subject matter of the present invention relates in a first aspect to a homoeostatic support system intended to apply an alternating cooling and heating sequence in at least one zone of a user's body surface, the system comprising:
a flexible support capable of being worn by the user;
control electronics; and
a software application,
   the support comprising at least one heating and cooling means and at least one temperature sensor intended to be associated with the zone, the control electronics being configured to receive data from the temperature sensor and to control the heating and cooling means, the application being configured to communicate by wired or wireless means with the control electronics so as to:
receive from the control electronics the temperature information measured by the sensor;
determine instructions for applying a given alternating cooling and heating sequence, according to the temperature information received, according to a program of the application; and
send the instructions to the control electronics.

In other words, the application of the system receives body temperature information in respect of a user, the information being obtained by a temperature sensor, for example a thermistor, and generates a set of instructions from the information received according to a program of the application, for example a program selected from a plurality of programs dedicated to muscle recovery, training, or injury or sprain treatment. The instructions are then sent to the control electronics, for example by wired means, the application being implemented by a microprocessor integrated in the control electronics, or by wireless means, the application being implemented remotely on a remote electronic device, for example a smartphone. The control electronics then apply the instructions so as to control the heating and cooling means associated with the flexible support worn by the user, resulting in an alternating cooling and heating sequence on the zone of the user's body surface.

It is understood here that the flexible support corresponds to a device intended to be worn by the user, in particular so as to embody a mobile device, its flexibility allowing it to adapt to the user's physical features and/or movements. The flexible support is for example made of a textile material. The control electronics are for example assembled on the support or connected thereto, in particular to the heating and cooling means of the support. In particular, the assembly of the control electronics on the support allows the embodiment of a portable assembly allowing the athlete to move. The control electronics are thus powered either on mains power, in particular via a transformer, in a simplified design with limited mobility, or on battery power in a portable design of the system.

It is additionally understood that the control electronics can be configured to control a plurality of heating and cooling means, for example disposed on several supports or on the same support. According to one variant, the control electronics are configured to control a single heating and cooling means. A person skilled in the art understands that such a selection is made according to the number and type of desired heating and cooling means, as well as the limitations of the control electronics, in particular the current required by the heating and cooling means with respect to the maximum current tolerated by the control electronics.

The person skilled in the art additionally understands that the alternating cooling and heating sequence successively generates vasoconstrictions and vasodilations having the effect of stimulating blood flow in the vicinity of the body surface receiving the heating and cooling means, according to a mechanism known as “vaso-pumping”, which allows a movement of metabolic substances (dioxygen, ions, nutrients), a reduction of the inflammatory response and its duration, facilitating repair of the exercised muscle and reducing metabolic processes in the muscle.

The Applicant thus proposes that such a solution makes it possible to create a rapid thermal shock using a portable device, without requiring additional external elements, for example ice or baths performing muscle cooling. Compared to these solutions, the thermal shock is also performed in an automated and controlled manner, making it possible to simplify and optimise use without requiring specific knowledge. The Applicant furthermore observes that the use of this solution makes it possible to obtain a significant improvement in the evacuation of lactic acid and the recovery of force production capabilities, allowing medium or long-term exercise repetition. This solution also has beneficial analgesic and anti-inflammatory effects in the context of acute pathologies, of surgical, trauma-related or rheumatological origin, for example muscle lesions, sprains, acute tendinopathies or in the context of post-surgical care or treatment of algoneurodystrophy.

Advantageously, the heating and cooling means is integrated inside the flexible support, the heating and cooling means also being flexible.

In other words, the heating and cooling means is associated with the flexible support so as to form a one-piece assembly. The flexible support corresponds for example to a textile support, the heating and cooling means being integrated in the flexible support for example by knitting and welding the electrical components with the flexible support.

In particular, the flexibility of the heating and cooling means inside the flexible support makes it possible to ensure good contact between the heating and cooling means and the zone of the user's body surface. This design thus makes it possible to improve heat transmission between the user's body and the heating and cooling means.

A person skilled in the art additionally understands that this design is optionally supplemented with cable guides integrated in the flexible support, so as to power the heating and cooling means and/or the control electronics, as well as to connect the heating and cooling means to the control electronics.

Thanks to the present invention, the homoeostatic support system allows the application of cooling and heating via the wearing of a mobile device, minimising the user's mobility and allowing use in varied scenarios, wherein cooling is performed without nitrogen, ice or liquid. The use of the system is therefore simplified and its efficiency increased compared to existing devices.

In an advantageous embodiment of the invention, at least one heating and cooling means includes at least one Peltier-effect cell.

Preferably, the control electronics includes an H-bridge configured to apply an alternating current through the Peltier-effect cell.

It is understood here that the Peltier-effect cell corresponds to a thermoelectric cooling module having two faces, the module making use of the Peltier effect so that the flow of a current through the Peltier-effect cell cools a first face of the module while heating a second face of the module. The current direction then defines whether the face of the Peltier cell associated with the zone of the user's body surface is cooled or heated. The use of the H-bridge then makes it possible to change the polarity of the current, i.e. invert the direction of the current so that the Peltier cell carries out the alternating cooling and heating sequence, according to the current flowing through it.

Advantageously, the Peltier-effect cell has a flexible structure, thus allowing it to be associated with the flexible support and to follow its deformation, in order to adapt the Peltier-effect cell to the zone of the user's body surface and/or its movements. The Peltier-effect cell is for example associated with a graphite layer forming a thermal conductor, or with any other material or element facilitating heat transfer from the Peltier-effect cell to the zone of the body surface.

Thus, the Peltier-effect cell makes it possible to embody a single heating and cooling means, controlled directly by the current flowing through it, the use of the H-bridge making it possible to alternate between cooling and heating.

Preferably, the control electronics include a voltage controller configured to apply a variable voltage and a constant current through the Peltier-effect cell.

The Applicant proposes that the use of such a voltage controller makes it possible to obtain a more linear operation of the Peltier-effect cell, in particular by keeping a constant electrical consumption, which makes it possible to achieve progressive and long-lasting cooling down while allowing the time required for heat dissipation on the other face of the Peltier-effect cell. For heating, the use of the voltage tester also makes it possible to obtain constant heating at a stabilised temperature, i.e. obtain better heating control.

Preferably, the voltage controller is controlled by pulse width modulation.

A person skilled in the art here understands that pulse width modulation (PWM) consists of a variation of the voltage pulse lengths, creating a variable voltage. The voltage controller, therefore the control electronics and the system more generally, is then more autonomous and can be controlled more easily, without requiring manual adjustment.

In a particular embodiment, the heating and cooling means is configured to apply a cooling sequence at less than 0° C., preferably between −10° C. and 0° C.

The Applicant observes that such a temperature of the cooling sequence makes it possible to cool body heat, during its use, to 10° C. In particular, the heating and cooling means is configured to perform cryotherapy, unlike mere cooling of the zone of the body surface.

In an additional embodiment, the flexible support comprises at least one heat dissipation means.

It is understood here that the heat dissipation means is configured to be associated with the heating and cooling means, for example with the Peltier-effect cell described above, which respectively generates and/or loses heat so as to cool or heat the zone of the user's body surface. Such a heat dissipation means thus makes it possible to evacuate and/or recover the heat generated and/or lost by the heating and cooling means, in particular outside the zone of the user's body surface, and therefore improve the heating and cooling applied on this zone.

Preferably, the at least one heat dissipation means comprises at least one fan, the control electronics being configured to control said fan.

The Applicant proposes that the use of such a fan makes it possible to improve the heating and cooling performance. In particular, in combination with a Peltier-effect cell as described above, the use of a fan allows the Peltier cell operating in cooling to reach negative temperatures according to the face associated with the zone of the user's body surface.

Of course, the at least one heat dissipation means may also comprise “passive” means requiring no control, for example the use of a heat sink facilitating convection cooling. Such a heat sink is for example also flexible so as to facilitate its wearing. According to a specific design, this heat sink is integrated with the fan in a one-piece element and/or removably assembled with a Peltier cell forming the heating and cooling means.

Thus, a textile support integrating a flexible Peltier-effect cell, associated with heat sinks on either side, the conductive wires of the Peltier-effect cell being integrated inside the flexible support, is provided in a particular design.

Preferably, the fan is arranged relative to the at least one heating and cooling means so as to expel air outwardly from the at least one heating and cooling means.

The Applicant observes that this arrangement of the fan, in particular unlike an arrangement aimed at blowing air towards the at least one heating and cooling means, makes it possible to improve heat dissipation and in particular to accelerate the temperature decrease. In particular, the combination of a Peltier-effect cell and a fan arranged so as to expel air from the Peltier-effect cell makes it possible to obtain, for use on a user, a cell temperature of 0° C. in less than 4 seconds.

In a specific embodiment, the at least one heat dissipation means comprises a plurality of micro-fans disposed adjacent to the heating and cooling means, the control electronics being configured to control the plurality of micro-fans.

The Applicant proposes that this design makes it possible to arrive at a flexible and high-performance assembly, in particular more flexible than one or more conventional fans and having a higher performance than a mere passive heat sink. The flexibility of the assembly also ensures that the micro-fans are disposed as close as possible to the heating and cooling medium in order to ensure their functionality.

For example, the micro-fans are also integrated inside the flexible support, like the heating and cooling means.

In an additional embodiment, the support further comprises at least one feedback means selected from a set of feedback means comprising:
a vibration device;
an electrostimulation device;
a shock wave generation device; and
a piezoelectric haptic feedback device,
   the control electronics being configured to control the at least one feedback means and the application being configured to communicate with the control electronics so as to send instructions for controlling the at least one feedback means.

It is understood here that the feedback means supplements the alternating cooling and heating sequence, so as to stimulate and control homoeostatic processes. In particular, the Applicant observes that the use of an electrostimulation device, in particular a transcutaneous electrical nerve stimulation device, known as TENS, has analgesic properties and facilitates the user's recovery. The feedback means is for example integrated with the heating and cooling means, or even juxtaposed thereto, so as to act on the same zone of the body surface. The program of the application is for example adapted so as to send variable instructions according to the presence and/or type of feedback means.

In a specific embodiment, the support further comprises at least one biosensor selected from a set of biosensors comprising:
a heart rate sensor;
a glucose level sensor;
a gluconic acid level sensor;
a lactic acid level sensor;
a pyruvic acid level sensor;
a potassium ion level sensor; and
a sodium ion level sensor,
   the control electronics being configured to receive data from the biosensor and the application being configured to receive biological information measured by the biosensor and to adapt the instructions sent according to the biological information.

It is understood here that the biosensor can be configured to measure the heart rate directly and thus estimate the blood flow, or the level of one or more ions or molecules regulated by the heart and respiration rate, in particular at the zone of the body surface receiving the heating and cooling means, and therefore at the muscles associated with this zone. The biosensor corresponds, for example, to an ion-selective electrode, known as an ISE (“Ion-Selective Electrode”, also known as “Specific Ion electrode”) making it possible to measure the concentration of a particular ion. The instructions determined by the application are then also determined according to the biological information returned by the biosensor.

A person skilled in the art furthermore understands that the glucose level makes it possible to assess local hypoglycaemia and/or hyperglycemia scenarios; that lactic acid, or lactate, is produced when the oxygen supply is insufficient and must be evacuated; that pyruvic acid, or pyruvate, is part of the glycolysis processes related to respiration and nutrient metabolism; that sodium ions (associated with hyponatremia and/or hypernatraemia) and potassium ions (associated with hypokalaemia and/or hyperkalaemia) are at central to the electrophysiological phenomena of muscles, in particular muscle contractions. Thus, the implementation of a thermal shock, carrying out “vaso-pumping”, improves the supply of oxygen, ions and nutrients as well as the evacuation of metabolic products. The use of additional sensors, and the adaptation of the program of the application to processing the information received by these sensors, thus makes it possible to fine-tune the use of the heating and cooling means, or the other feedback means described above, in order to guarantee optimal muscle performance.

In another embodiment, the support further comprises at least one surface electromyography sensor, the control electronics being configured to receive data from the surface electromyography sensor and the application being configured to receive neuromuscular information measured by the surface electromyography sensor and to adapt the instructions sent according to the neuromuscular information.

A person skilled in the art here understands that surface electromyography, known as EMG, allows a non-invasive analysis of the neuromuscular system, the surface EMG sensor corresponding for example to one or more electrodes disposed on the skin of the zone of the user's body surface. The neuromuscular information measured by the EMG sensor can thus be used to monitor muscle activity, detect sensitive zones and fatigue thresholds, in order to optimise the recovery process controlled by the application.

A second aspect of the present invention relates to a homoeostatic support suit intended to apply an alternating cooling and heating sequence in at least one zone of a user's body surface, the suit comprising at least one sleeve receiving a support connected to control electronics of the thermoregulation system according to the first aspect of the invention, the heating and cooling means and the temperature sensor of the support being disposed in the suit so as to be associated with the zone when the user wears the suit.

It is understood here that the suit embeds the flexible support and the control electronics of the system according to the first aspect of the invention, the control electronics communicating with the application by wired or wireless means, for example communicating remotely with a remote electronic device independent of the suit. The suit includes, for example, a plurality of sleeves, making it possible to treat large body surfaces or the entire body. According to a particular design, the sleeves are disposed according to a map of the motor points of the body, so as to target the action of the suit on all the zones of the body to be treated to optimise the user's physical recovery. Integrating the homoeostatic support system in a suit also facilitates the user's mobility

Preferably, the suit has a compressive effect according to the at least one zone of the user's body surface.

It is understood here that compression, for example dynamic, supplements the heating and cooling action, for example like the feedback means described above, by supporting “vaso-pumping”. The compressive effect is, for example, adjustable between 0 and 75 mm of mercury.

Thus, by the different functional and structural technical features above, the Applicant proposes a homoeostatic support system and suit allowing cooling and heating of a zone of the body surface using a portable device, making it possible to improve recovery capabilities and prevent fatigue-related lesions and injuries, and applying cooling without ice or liquid baths.

### BRIEF DESCRIPTION OF THE FIGURES

The features of the present description will become apparent from the description above with reference to appended FIGS. 1 to 6 illustrating a plurality of example embodiments which are devoid of any limiting nature and wherein:

FIG. 1 shows a schematic view of a homoeostatic support system according to an example embodiment of the present invention;

FIG. 2 shows a schematic exploded diagram of a first example embodiment of a flexible support integrated in a system according to FIG. 1;

FIG. 3 shows a schematic view of a second example embodiment of a flexible support integrated in a system according to FIG. 1;

FIG. 4 shows a schematic view of a first example embodiment of a Peltier-effect cell integrated in a flexible support of a system according to FIG. 1;

FIG. 5 shows a flowchart of the different steps of a homoeostatic support method implemented by a system according to FIG. 1;

FIG. 6 shows a schematic view of a second example embodiment of a Peltier-effect cell integrated in a flexible support of a system according to FIG. 1.

### DETAILED DESCRIPTION

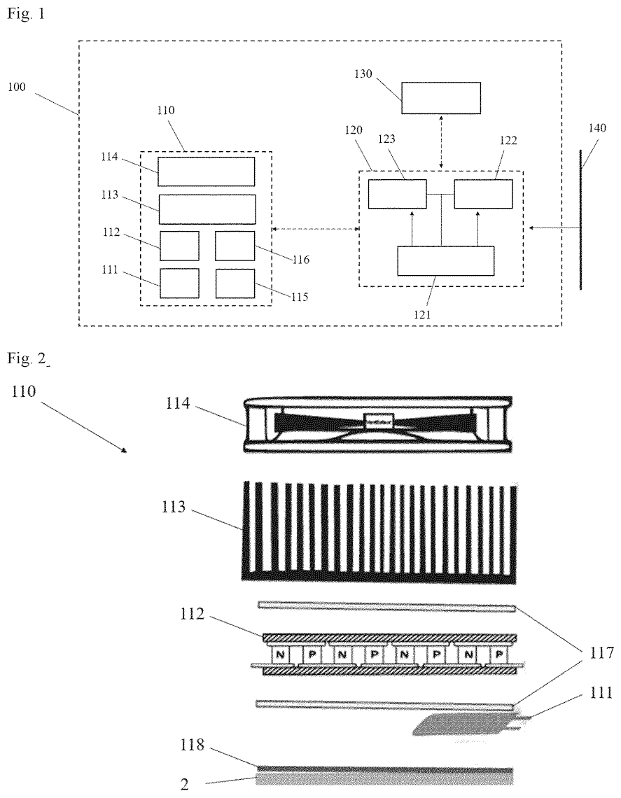

The present invention will now be described hereinafter with reference jointly to FIGS. 1 to 6 appended to the description.

As stated in the preamble of the description, current homoeostatic support solutions are generally cumbersome and allow only limited recovery after exertion.

One of the objectives of the present invention consists of allowing the use, by an athlete or any other user, for example a person in rehabilitation or regularly practising a sports discipline, of a system facilitating their muscle recovery and having limited dimensions.

This is enabled in the example described hereinafter.

According to the example of FIG. 1, a homoeostatic support system 100 developed within the scope of the present invention is employed by a user, for example an athlete. In a specific design, the user wears a homoeostatic support suit, i.e. a garment, for example a vest or even an all-in-one suit, which integrates the hardware elements of the system 100. The suit includes for example one or more sleeves, i.e. a pocket or portion of the suit, receiving the hardware elements of the system 100. In particular, the positioning of the sleeve(s), i.e. the arrangement of the hardware elements of the system 100, makes it possible to define at least one zone 2 of the users body surface on which the system 100 acts. Thus, the sleeve(s) of the suit are for example advantageously disposed so as to cover the motor points of the user's body, in particular according to a given map of the motor points.

Optionally, the suit has a compressive effect according to the zone 2, i.e. it is configured to apply pressure on the user's body surface. In particular, an adjustable pressure, between 0 and 75 mm of mercury, i.e. between 0 and 0.1 bar, or even a dynamic pressure that changes over time, helps the user's blood flow along the zone 2, and thus controls homoeostasis via the supply of dioxygen, ions and nutrients and the evacuation of muscle activity products.

In the same example, the system 100 comprises a flexible support 110 capable of being worn by the user, the support 110 comprises at least one heating and cooling means 112 and at least one temperature sensor 111, for example a thermistor, intended to be associated with the zone 2; the support 110 constitutes at least a part of the hardware elements of the system 100, as set out above. The flexible support 110 is thus disposed so as to act on the zone 2 of the user's body surface. According to the example of FIG. 2, the support 110 also comprises a film 118 coming into direct contact with the zone 2, the temperature sensor 111 and the heating and cooling means 112 being disposed on this film 118. The film 118 corresponds for example to a graphene film, embodying a light and flexible support 110 adapting to the shape of the zone 2, i.e. to the user's physical features and movement.

In combination with the support 110, the system 100 includes control electronics 120 in communication with the temperature sensor 111, so as to receive the data thereof, and with the heating and cooling means 112, so as to control it. For example, the control electronics 120 are also integrated in the suit, without for all that needing to be in direct contact with the zone 2. The control electronics 120 can thus be arranged on the suit so as to facilitate wearing it, or even to be connected to several separate supports 110, for example arranged along several sleeves of the suit. As a general rule, the control electronics 120 are thus advantageously configured to power and control the electronic elements of the support 110. In this respect, the control electronics 120 are supplied with electricity by a source 140, for example a source internal to the system 100 and worn by the user, in particular a battery in a portable design of the system 100 facilitating the user's movement and autonomy, or even a source external to the system 100, corresponding in particular to a transformer sized for powering the control electronics 140, the system 100 then operating when connected on mains power. In order to enable the control of the electronic elements of the support 110, the control electronics 120 thus comprise at least one processor 121.

Finally, the system 100 also comprises a software application configured to communicate with the control electronics 120 via wired or wireless means. According to a first design, the software application is directly implemented by the processor 121, the latter communicating with the other elements of the control electronics by wired means. According to a second design, the software application is implemented by a remote device 130 in communication with the control electronics 120, in particular with the processor 121. This design makes it possible in particular to limit the processing capacities required by the processor 121 and to replace them with communication means with the remote device 130, so as to limit the overall dimensions generated by the control electronics 120. The remote device 130 corresponds for example to a smartphone of the user, communicating with the control electronics by wired or wireless means, for example by Bluetooth. The software application and the control electronics 120 thus make it possible to control the heating and cooling means 112 and to receive data from the temperature sensor 111.

According to the examples of FIGS. 2 to 4 and 6, the at least one heating and cooling means 112 includes at least one Peltier-effect cell, i.e. a cell having two faces and configured to receive a current circulating through the cell, the circulation of the current generating cooling of a first face and heating of a second face opposite the first face, by applying the Peltier effect. In combination with such a Peltier-effect cell, the control electronics 120 includes an H-bridge 122 configured to apply an alternating current through the Peltier-effect cell. In other words, the processor 121 controls the operation of the H-bridge 122 so as to send a current through the Peltier-effect cell, the polarity of the current changing the direction wherein the Peltier effect takes place, i.e. exchanging the cooling and heating of the first and second face.

Thus, the Peltier-effect cell makes it possible to carry out, in a single element, alternating heating and cooling, according to the control performed by the control electronics 120.

The example in FIGS. 4 and 6 illustrates in more detail a Peltier-effect cell according to an alternative embodiment of the invention. In this example, the Peltier-effect cell has a first conductive wire 112a and a second conductive wire 112b, each being assembled with two heat sinks 112c forming the two faces of the Peltier-effect cell, for example via an assembly of welds 112d. The first conductive wire 112a corresponds for example to the phase and the second conductive wire 112b to the neutral of the Peltier-effect cell. Advantageously, a flexible thermal insulator 112e, i.e. a conductive material in which the thermoelectric power is assumed to be zero, is disposed between the two heat sinks 112c, making it possible to separate the two faces of the Peltier cell and thus improve its efficiency. According to a particular design, the Peltier-effect cell is integrated in the flexible support 110, the Peltier-effect cell also having a flexible structure. For example, the flexible support 110 forms the flexible thermal insulator 112e, the conductive wires 112a, 112b being knitted with the thermal insulator 112e so as to dispose the heat sinks 112c on either side of the surface of the flexible support 110. The textile structure allowing the integration of a flexible heating and cooling means 112 corresponds for example to a velvet fabric, a 3D weave, a warp-stitch knit, or insertion of the thermoelectric wires by stitching. This design thus makes it possible to fully integrate the heating and cooling means 112 with the flexible support 110 unlike a simple assembly, and to dispose the heating and cooling means 112 directly in contact with the zone 2 of the user's body surface when using the system 100.

Advantageously, and as illustrated in FIG. 2, an electrical insulating film 117 disposed on either side of the Peltier-effect cell is provided, allowing its operation without risk to the user or the other components of the support 110.

In the example of FIG. 1, a voltage controller 123 integrated in the control electronics 120 and associated with the H-bridge 122 is additionally provided so as to power the Peltier-effect cell. Such a voltage controller 123 is advantageously configured to apply a variable voltage and a constant current through the Peltier-effect cell, resulting in a more linear evolution of the temperature of the Peltier-effect cell, facilitating heat dissipation and allowing a better hold of the sought temperature over time. For example, the variable voltage of the voltage controller 123 is obtained via a control by pulse width modulation, making it possible to approximate a linear evolution of the voltage.

Optionally, and as illustrated in FIGS. 2, 3 and 6, the flexible support additionally comprises at least one heat dissipation means 113, 114, 114′. In these examples, the heat dissipation means 113, 114, 114′ makes it possible to improve heat exchanges with the face of the Peltier-effect cell which does not act on the zone 2 of the user's body surface. The temperature gradient inside the heating and cooling means 112 is therefore reduced, limiting internal heat transfers and improving its performance. A passive heat sink 113 is thus provided, in particular a device provided with a plurality of fins mounted vertically with respect to the heating and cooling means 112, thereby embodying a passive heat sink 113 having a large contact surface with the outside air and increasing heat exchanges by convection. A fan 114 is also provided, for example combined with the passive heat sink as illustrated in FIG. 2, the control electronics 120 also controlling the actuation of the fan 114. Preferably, the fan 114 is arranged so as to blow the air outwardly from the heating and cooling means 112, and here from the passive heat sink 113, i.e. in the direction opposite the zone 2 of the user's body surface. In the example of FIG. 6, a plurality of micro-fans 114′ are provided, in replacement of or in addition to the passive heat sink 113 and/or the fan 114. The micro-fans 114′ are advantageously disposed adjacently to the heating and cooling means 112. For example, the micro-fans are also arranged so as to blow air outwardly from the heating and cooling means 112, so as to obtain more efficient cooling.

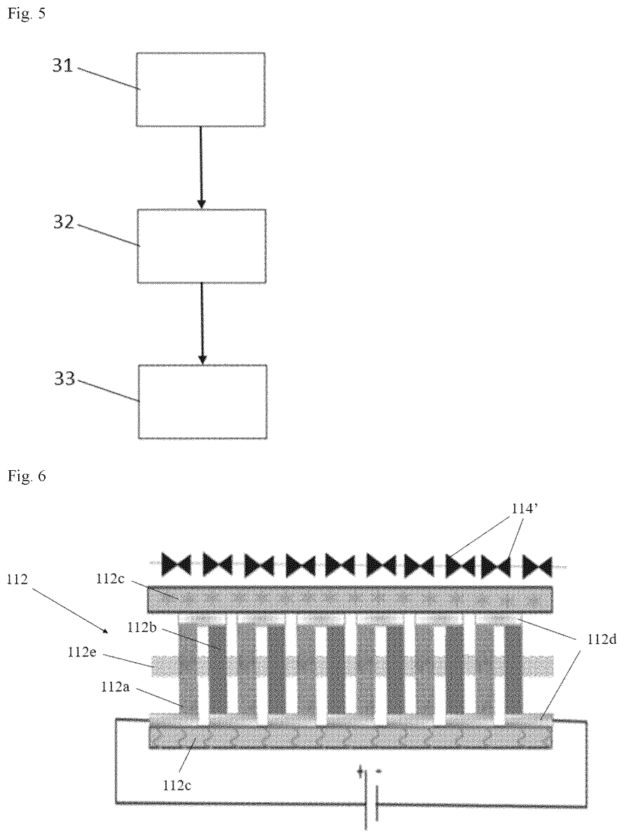

The system described above is thus configured to implement the steps of a homoeostatic support method according to FIG. 5. In a first step 31, the application receives from the control electronics 120 temperature information measured by the temperature sensor 111, i.e. the data from the temperature sensor 111 and received by the control electronics 120 are sent for processing according to the application. In accordance with the above description, the information is thus sent to the processor 121 and/or the remote device 130.

In a second step 32, the application determines instructions for applying a defined alternating cooling and heating sequence according to the temperature information received. The instructions are also determined according to a program of the application, for example a targeted program for strength training, quick recovery, rehabilitation or any other program, making it possible to adapt the instructions to different use scenarios or to different user profiles.

The instructions are then sent in a third step 33 to the control electronics 120, which implement them, in particular by controlling the heating and cooling means 112 and optionally the fan 114 and/or the micro-fans 114′ described above, so as to optimise the operation of the heating and cooling means 112.

Thus, the heating and cooling means 112 is controlled so as to carry out an alternating heating and cooling sequence on the zone 2 of the user's body surface. This sequence results in a plurality of vasoconstrictions and vasodilations, generating a “vaso-pumping” mechanism stimulating muscular blood flow and facilitating energy recovery and the supply of nutrients, ions and dioxygen.

Optionally, an additional intermediate step is provided wherein the temperature information received is compared to at least one threshold value, the instructions determined during the second step 32 being adapted according to a result of the comparison. In particular, the program of the software application is configured to generate an inversion between heating and cooling according to the result, in particular so as to control heating when the temperature information is less than a first threshold value, and cooling when the temperature information is greater than a second threshold value. Such operation makes it possible to ensure additional safety for the system during use, in particular to prevent burns resulting from extreme temperatures.

Advantageously, the heating and cooling means 112, for example the Peltier-effect cell described above and associated with the heat dissipation means 113, 114, 114′ is configured to perform cooling below 0° C., preferably between −10° C. and 0° C., such a temperature making it possible to perform cryotherapy, lowering the body heat of the zone 2 to 10° C. Such a temperature reduction makes it possible to ensure effective vasoconstriction and to differentiate the action of the heating and cooling means 112 from mere cooling of the zone 2.

Optionally, the support 110 also comprises at least one biosensor 115, the control electronics 120 receiving the data from the biosensor 115 and the application being configured to receive, during the first step 31, biological information measured by the biosensor 115, the application also being configured to adapt, during the second and third steps, the information determined and sent according to the biological information.

The biosensor 115 corresponds for example to a heart rate sensor, thus making it possible to determine the blood flow occurring in the muscle associated with the zone 2, or to one or more sensors measuring an ion or nutrient level in the user's blood, in particular to an ion-selective electrode. The biosensor 115 thus makes it possible, for example, to measure a glucose level in order to determine whether the sugar supply is too low or too high, i.e. hypoglycaemia or local hyperglycemia of the zone 2, a level of gluconic, lactic or pyruvic acid produced during glycolysis and to be evacuated from the muscles associated with the zone 2, or a potassium or sodium ion level in order to detect hypokalaemia, hyperkalaemia, hyponatraemia and hypernatraemia scenarios, i.e. monitor and regulate the ion concentration allowing proper muscle contraction.

Thus, according to a particular design, the information received from the biosensor 115 is processed by the application so as to regulate the rhythm of the alternating cooling and heating sequence, in order to accelerate or slow down the blood flow at the muscle associated with the zone 2, the information received from the temperature sensor 111 being processed so as to monitor the cooling and heating applied according to the determined sequence, so as to confirm that the cooling and heating means 112 has the intended effect.

According to another variant, the support 110 comprises one or more surface electromyography sensors, referred to as EMG sensor, the control electronics 120 receiving the data from the EMG sensor and the application receiving neuromuscular information measured by the EMG sensor during the first step 31 and adapting the instructions of the second and third steps 32, 33 according to the neuromuscular information. According to a principle similar to that described above with regard to the biosensors 115, the use of EMG sensors makes it possible to specify the action of the cooling and heating means 112 and to adapt it to the needs of the muscle treated, in particular by detecting signs of muscle fatigue.

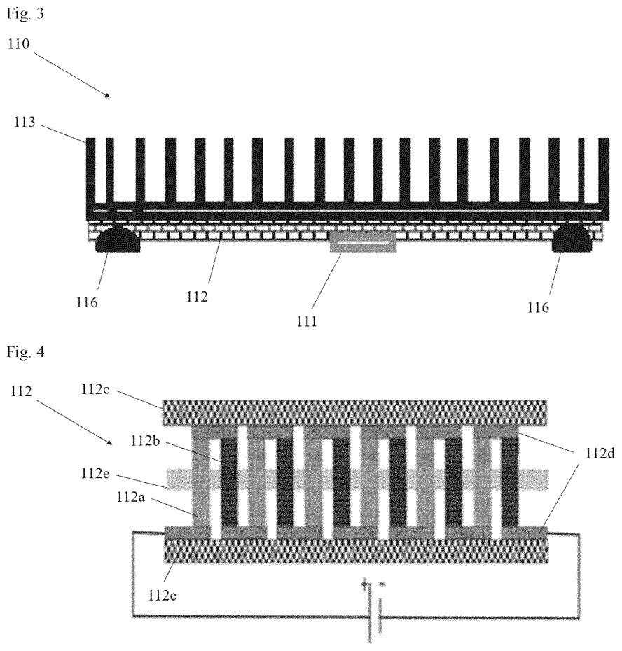

According to yet another variant illustrated in FIG. 3, the support 110 also comprises a feedback means 116, for example disposed adjacently to the temperature sensor 111, in contact with the zone 2 of the user's body surface. Such a feedback means 116 corresponds for example to a vibration device, an electrostimulation device, a shock wave generation device or a piezoelectric haptic feedback device. The instructions determined and sent by the application during the second and third steps 32, 33 then also comprise instructions for controlling the feedback means 116. The action of the feedback means 116 then supplements the “vaso-pumping” implemented by the cooling and heating means 112 and/or provides an additional homoeostatic support means in parallel with the cooling and heating means 112.

Thus, it will be understood that the present invention provides a homoeostatic support system, and a suit at least partially integrating such a system, the system allowing the application of an alternating cooling and heating sequence on at least one zone of a user's body surface, thus generating both vasoconstriction and vasodilations within a single controlled assembly according to an application, without requiring baths or ice to supplement the action. Preferably, cooling and heating are implemented using at least one Peltier-effect cell enabling these two actions to be performed using a simple and compact device. Such a system can also be supplemented by the use of additional sensors and feedback means, in order to refine the behaviour of the system and optimise muscle recovery, in particular according to the precise needs of the muscle treated.

It should be observed that this detailed description concerns a particular example embodiment of the present invention, but that this description in no way applies any limiting nature to the subject matter of the invention; on the contrary, it is intended to remove any inaccuracy or any incorrect interpretation of the following claims.

It should also be observed that the reference signs placed between parentheses in the following claims are in no way limiting; these signs are merely intended to improve the intelligibility and comprehension of the following claims as well as the scope of the protection sought.

1. A homeostatic support system intended to apply an alternating cooling and heating sequence in at least one zone of a user's body surface, said system comprising:
a flexible support capable of being worn by said user, the flexible support comprising at least one heating and cooling means and at least one temperature sensor intended to be associated with the at least one zone;
control electronics configured to receive data from the temperature sensor and to control the heating and cooling means; and
a software application configured to communicate by wired or wireless means with the control electronics so as to:
receive from said control electronics temperature information measured by said temperature sensor;
determine instructions for applying a given alternating cooling and heating sequence, according to said temperature information received, according to a program of the application; and
send said instructions to said control electronics,
wherein said heating and cooling means is are flexible and are integrated inside said flexible support.

2. The homeostatic support system according to claim 1, wherein said at least one heating and cooling means comprises at least one Peltier-effect cell, said control electronics including an H-bridge configured to apply an alternating current through said Peltier-effect cell.

3. The homeostatic support system according to claim 2, wherein said control electronics include a voltage controller configured to apply a variable voltage and a constant current through said Peltier-effect cell.

4. The homeostatic support system according to claim 3, wherein said voltage controller is controlled by pulse width modulation.

5. The homeostatic support system according to claim 1, wherein said heating and cooling means is configured to apply a cooling sequence at less than 0° C., preferably between −10° C. and 0° C.

6. The homeostatic support system according to claim 1, wherein said flexible support comprises at least one heat dissipation means.

7. The homeostatic support system according to claim 6, wherein said at least one heat dissipation means comprises at least one fan, said control electronics being configured to control said fan.

8. The homeostatic support system according to claim 7, wherein said fan is arranged relative to said at least one heating and cooling means so as to expel air outwardly from said at least one heating and cooling means.

9. The homeostatic support system according to claim 6, wherein said at least one heat dissipation means comprises a plurality of micro-fans disposed adjacent to said heating and cooling means, said control electronics being configured to control said plurality of micro-fans.

10. The homeostatic support system according to claim 1, wherein said support further comprises at least one feedback means selected from a group of feedback means consisting of a vibration device, an electrostimulation device, a shock wave generation device and a piezoelectric haptic feedback device,
said control electronics being configured to control said at least one feedback means and said application being configured to communicate with said control electronics so as to send instructions for controlling said at least one feedback means.

11. The homeostatic support system according to claim 1, wherein said support further comprises at least one biosensor selected from a group of biosensors consisting of a heart rate sensor, a glucose level sensor, a gluconic acid level sensor, a lactic acid level sensor, a pyruvic acid level sensor, a potassium ion level sensor, and a sodium ion level sensor,
said control electronics being configured to receive data from said at least one biosensor and said application being configured to receive biological information measured by said at least one biosensor and to adapt said instructions sent according to said biological information.

12. The homeostatic support system according to claim 1, wherein said support further comprises at least one surface electromyography sensor, said control electronics being configured to receive data from said at least one surface electromyography sensor and said application being configured to receive neuromuscular information measured by said at least one surface electromyography sensor and to adapt said instructions sent according to said neuromuscular information.

13. A homeostatic support suit intended to apply an alternating cooling and heating sequence in at least one zone of a user's body surface, said suit comprising:
at least one sleeve receiving a support connected to control electronics of the homoeostatic support system according to claim 1, said heating and cooling means and said temperature sensor of said support being disposed in the homeostatic support suit so as to be associated with said zone when the user wears the homeostatic support suit.

14. The homeostatic support suit according to claim 13, wherein it has a compressive effect according to said at least one zone of said user's body surface.
